(19) United States
(12) Patent Application Publication
(10) Pub. No.: US 2008/0227207 A1
(43) Pub. Date: Sep. 18, 2008

(54) AUTOMATED COLORIMETRIC POLYSACCHARIDE ASSAYS

(75) Inventors: Vincent E. Turula, Holly Springs, NC (US); Shaune Walters, Raleigh, NC (US); Suddham Singh, Chapel Hill, NC (US); Rasappa Arumugham, Chapel Hill, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Wyeth, Madison, NJ (US)

(21) Appl. No.: 12/048,010

(22) Filed: Mar. 13, 2008

Related U.S. Application Data
(60) Provisional application No. 60/894,796, filed on Mar. 14, 2007.

(51) Int. Cl. G01N 33/50

(52) U.S. Cl. 436/43

(57) ABSTRACT

A method of automated determination of saccharide concentration is provided herein. The method includes preparing one or more saccharide standards and one or more diluted polysaccharide test samples, transferring a portion of the one or more saccharide standards and the one or more diluted polysaccharide test samples along with a diluent to a series of wells in a multiwell plate, transferring a portion of an acid reagent to the series of wells in the multiwell plate, mixing the contents of the series of wells in the multiwell plate, heating and cooling the contents of the series of wells in the multiwell plate, shaking the multiwell plate, and measuring the radiant energy absorbance of the contents of the series of wells in the multiwell plate, where all steps are performed in the absence of human intervention.

### CROSS-REFERENCE TO RELATED APPLICATIONS

This application claims the benefit of U.S. Provisional Patent Application No. 60/894,796, filed Mar. 14, 2007, which is hereby incorporated in its entirety by reference herein.

### FIELD OF THE INVENTION

The present invention relates to automated methods for determining saccharide concentration.

### BACKGROUND OF THE INVENTION

Measurement of saccharide content in a variety of samples is a basic analytical operation in many areas of science. Well known calorimetric methods for saccharide concentration determination include assays employing anthrone (Dreywood, R., Ind. Eng. Chem., Anal. Ed. 18:499, 1946; Morse, E. E., Anal. Chem. 19:1012, 1947; and Morris, D. L., Science 107:254, 1948), phenol reagents (Dubois et al, Nature 168:167, 1951; Dubois et al., Anal. Chem. 28:350-56, 1956; and Blumenkrantz and Asboe-Hansen, Anal. Biochem. 54:484, 1973), orcinol (Irwin and Leaver, Nature 177:1126, 1956), and resorcinol (Monsigny et al., Anal. Biochem. 175:525-30, 1988). The anthrone assay is used to measure the concentration of saccharides in polysaccharides that contain neutral hexoses, while phenol reagents (e.g., meta-hydroxydiphenyl (mHDP)/3-phenylphenol) are used to measure the concentration of saccharides in polysaccharides that contain galacturonic or glucouronic acid monosaccharides.

Sources of variability within the various calorimetric assays for saccharide concentration determination include elements related to sample preparation and measurement by a human operator, such as sample handling, reagent dilutions, instability of reagents, variation of heating and cooling times, and the like. Sampling difficulties are inherent in the application of many of these assays, as dilutions from mg/ml to μg/ml concentrations are often needed to bring samples to a quantitatable level relative to standards. Despite rigorous protocols, sampling difficulties, namely the need for human operators to dilute concentrated solutions to very dilute solutions, prevents these assays from evolving into accurately reliable assays from which formulation into drug product and label claims are made. Therefore, there remains a need for the development of effective automated methods for determining saccharide concentration.

### SUMMARY OF THE INVENTION

A method of automated determination of saccharide concentration is provided. The method includes: (1) preparing one or more saccharide standards, including the steps of (a) transferring a portion of a saccharide working stock solution from a saccharide working stock solution source container to multiple saccharide standard receiving containers, (b) transferring a portion of a diluent from a diluent source container to the multiple saccharide standard receiving containers, and (c) mixing the contents of the multiple saccharide standard receiving containers to prepare the one or more saccharide standards; (2) preparing one or more diluted polysaccharide test samples, including the steps of (a) transferring a portion of a polysaccharide test sample from a polysaccharide test sample source container to multiple polysaccharide test sample receiving containers, (b) transferring a portion of the diluent from the diluent source container to the multiple polysaccharide test sample receiving containers, and (c) mixing the contents of the multiple polysaccharide test sample receiving containers to prepare the one or more diluted polysaccharide test samples; (3) transferring a portion of the diluent from the diluent source container to a series of wells in a multiwell plate; (4) transferring a portion of the one or more saccharide standards from each of the multiple saccharide standard receiving containers to a series of wells in the multiwell plate; (5) transferring a portion of the one or more diluted polysaccharide test samples from each of the multiple polysaccharide test sample receiving containers to a series of wells in the multiwell plate; (6) transferring a portion of an acid reagent from an acid reagent source container to all of the series of wells in the multiwell plate containing the diluent, the one or more saccharide standards, and the one or more diluted polysaccharide test samples; (7) mixing the contents of all of the series of wells in the multiwell plate containing the diluent, the one or more saccharide standards, and the one or more diluted polysaccharide test samples; (8) covering the multiwell plate with a cover; (9) heating the multiwell plate; (10) cooling the multiwell plate; (11) shaking the multiwell plate; and (12) measuring the radiant energy absorbance of the contents of all of the series of wells in the multiwell plate containing the diluent, the one or more saccharide standards, and the one or more diluted polysaccharide test samples, where steps 1-12 are performed without human intervention.

In one embodiment, an additional step of transferring a portion of a color reagent from a color reagent source container to all of the series of wells in the multiwell plate containing the diluent, the one or more saccharide standards, and the one or more diluted polysaccharide test samples, is performed after cooling the multiwell plate.

Polysaccharide test samples to be assayed for saccharide concentration using the methods of the invention include test samples containing a bacterial capsular polysaccharide, an activated bacterial capsular polysaccharide or a bacterial capsular polysaccharide-protein conjugate. Exemplary bacterial capsular polysaccharides include bacterial capsular polysaccharides from N. meningitidis (Nm) serogroups Y and W₁₃₅; Group B Streptococcus (GBS) serotypes Ia and III; and S. pneumoniae serotypes 1, 3, 4, 5, 6A, 6B, 7F, 9V, 14, 18C, 19A, 19F, and 23F.

In another embodiment, the method includes: (1) preparing one or more saccharide standards, including the steps of (a) transferring a portion of a saccharide working stock solution including one or more saccharides selected from the group consisting of rhamnose, galactose, glucose, galacturonic acid, pyruvic acid, N-acetyl-D-galactosamine, and N-acetyl-D-mannosamine from a saccharide working stock solution source container to multiple saccharide standard receiving containers, (b) transferring a portion of a diluent from a diluent source container to the multiple saccharide standard receiving containers, and (c) mixing the contents of the multiple saccharide standard receiving containers to prepare the one or more saccharide standards; (2) preparing one or more diluted polysaccharide test samples, including the steps of (a) transferring a portion of a polysaccharide test sample including a bacterial capsular polysaccharide selected from a group of bacteria consisting of S. pneumoniae serotypes 3, 4, 6A, 6B, 7F, 9V, 14, 18C, 19A, 19F, and 23F from a polysaccharide test sample source container to multiple polysaccharide test sample receiving containers, (b) transferring a portion of the diluent from the diluent source container to the multiple polysaccharide test sample receiving containers, and (c) mixing the contents of the multiple polysaccharide test sample receiving containers to prepare the one or more diluted polysaccharide test samples; (3) transferring a portion of the diluent from the diluent source container to a series of wells in a multiwell plate; (4) transferring a portion of the one or more saccharide standards from each of the multiple saccharide standard receiving containers to a series of wells in the multiwell plate; (5) transferring a portion of the one or more diluted polysaccharide test samples from each of the multiple polysaccharide test sample receiving containers to a series of wells in the multiwell plate; (6) transferring a portion of a sulfuric acid-anthrone reagent from a sulfuric acid-anthrone reagent source container to all of the series of wells in the multiwell plate containing the diluent, the one or more saccharide standards, and the one or more diluted polysaccharide test samples; (7) mixing the contents of all of the series of wells in the multiwell plate containing the diluent, the one or more saccharide standards, and the one or more diluted polysaccharide test samples; (8) covering the multiwell plate with a cover; (9) heating the multiwell plate; (10) cooling the multiwell plate; (11) shaking the multiwell plate; and (12) measuring the radiant energy absorbance of the contents of all of the series of wells in the multiwell plate containing the diluent, the one or more saccharide standards, and the one or more diluted polysaccharide test samples, where steps 1-12 are performed without human intervention.

In a further embodiment, the method includes: (1) preparing one or more saccharide standards, including the steps of (a) transferring a portion of a saccharide working stock solution including galacturonic acid from a saccharide working stock solution source container to multiple saccharide standard receiving containers, (b) transferring a portion of a diluent from a diluent source container to the multiple saccharide standard receiving containers, and (c) mixing the contents of the multiple saccharide standard receiving containers to prepare the one or more saccharide standards; (2) preparing one or more diluted polysaccharide test samples, including the steps of (a) transferring a portion of a polysaccharide test sample including a bacterial capsular polysaccharide selected from a group of bacteria consisting of S. pneumoniae serotypes 1 and 5 from a polysaccharide test sample source container to multiple polysaccharide test sample receiving containers, (b) transferring a portion of the diluent from the diluent source container to the multiple polysaccharide test sample receiving containers, and (c) mixing the contents of the multiple polysaccharide test sample receiving containers to prepare the one or more diluted polysaccharide test samples; (3) transferring a portion of the diluent from the diluent source container to a series of wells in a multiwell plate; (4) transferring a portion of the one or more saccharide standards from each of the multiple saccharide standard receiving containers to a series of wells in the multiwell plate; (5) transferring a portion of the one or more diluted polysaccharide test samples from each of the multiple polysaccharide test sample receiving containers to a series of wells in the multiwell plate; (6) transferring a portion of a sulfuric acid-tetraborate reagent from a sulfuric acid-tetraborate reagent source container to all of the series of wells in the multiwell plate containing the diluent, the one or more saccharide standards, and the one or more diluted polysaccharide test samples; (7) mixing the contents of all of the series of wells in the multiwell plate containing the diluent, the one or more saccharide standards, and the one or more diluted polysaccharide test samples; (8) covering the multiwell plate with a cover; (9) heating the multiwell plate; (10) cooling the multiwell plate; (11) transferring a portion of a color reagent including meta-hydroxydiphenyl from a color reagent source container to all of the series of wells in the multiwell plate containing the diluent, the one or more saccharide standards, and the one or more diluted polysaccharide test samples; (12) shaking the multiwell plate; and (13) measuring the radiant energy absorbance of the contents of all of the series of wells in the multiwell plate containing the diluent, the one or more saccharide standards, and the one or more diluted polysaccharide test samples, where steps 1-13 are performed without human intervention.

Using the methods of the invention improves the accuracy of determining saccharide concentration, as elements of traditional assays for saccharide concentration determination related to sample preparation and measurement by a human operator (e.g., sample handling and reagent dilutions) are eliminated.

### DETAILED DESCRIPTION OF THE INVENTION

The present invention provides a method directed to automated determination of saccharide concentration. Specifically, the method includes: (1) preparing one or more saccharide standards, including the steps of (a) transferring a portion of a saccharide working stock solution from a saccharide working stock solution source container to multiple saccharide standard receiving containers, (b) transferring a portion of a diluent from a diluent source container to the multiple saccharide standard receiving containers, and (c) mixing the contents of the multiple saccharide standard receiving containers to prepare the one or more saccharide standards; (2) preparing one or more diluted polysaccharide test samples, including the steps of (a) transferring a portion of a polysaccharide test sample from a polysaccharide test sample source container to multiple polysaccharide test sample receiving containers, (b) transferring a portion of the diluent from the diluent source container to the multiple polysaccharide test sample receiving containers, and (c) mixing the contents of the multiple polysaccharide test sample receiving containers to prepare the one or more diluted polysaccharide test samples; (3) transferring a portion of the diluent from the diluent source container to a series of wells in a multiwell plate; (4) transferring a portion of the one or more saccharide standards from each of the multiple saccharide standard receiving containers to a series of wells in the multiwell plate; (5) transferring a portion of the one or more diluted polysaccharide test samples from each of the multiple polysaccharide test sample receiving containers to a series of wells in the multiwell plate; (6) transferring a portion of an acid reagent from an acid reagent source container to all of the series of wells in the multiwell plate containing the diluent, the one or more saccharide standards, and the one or more diluted polysaccharide test samples; (7) mixing the contents of all of the series of wells in the multiwell plate containing the diluent, the one or more saccharide standards, and the one or more diluted polysaccharide test samples; (8) covering the multiwell plate with a cover; (9) heating the multiwell plate; (10) cooling the multiwell plate; (11) shaking the multiwell plate; and (12) measuring the radiant energy absorbance of the contents of all of the series of wells in the multiwell plate containing the diluent, the one or more saccharide standards, and the one or more diluted polysaccharide test samples, where steps 1-12 are performed without human intervention.

By “saccharide standards” is intended a series of standards with known saccharide concentrations in each standard of the series. Such a series of standards can include a range of concentrations of saccharides, for example from 2.5 nM to 100 nM, such as 2.5 nM, 5 nM, 10 nM, 15 nM, 20 nM, 25 nM, 30 nM, 35 nM, 40 nM, 45 nM, 50 nM, 55 nM, 60 nM, 65 nM, 70 nM, 75 nM, 80 nM, 85 nM, 90 nM, 95 nM, and 100 nM. Theoretical standard concentrations (e.g., nM) are plotted versus measured mean absorbance of a set of replicates of each standard concentration in the series, and the slope, y-intercept and correlation of coefficient (r) are calculated by linear regression analysis to produce a standard curve.

As used herein, the term “saccharide working stock solution” includes a solution having a saccharide composition that reflects the composition of a polysaccharide repeat unit from the capsular polysaccharide of a particular bacterium. Exemplary bacteria include N. meningitidis serogroup Y (glucose repeat unit), N. meningitidis serogroup W₁₃₅ (galactose repeat unit), Group B Streptococcus serotype Ia (glucose/galactose repeat unit), Group B Streptococcus serotype III (glucose/galactose repeat unit), S. pneumoniae serotype 1 (galacturonic acid repeat unit), S. pneumoniae serotype 3 (glucose repeat unit), S. pneumoniae serotype 4 (galactose/N-acetyl-D-galactosamine/N-acetyl-D-mannosamine/pyruvic acid repeat unit), S. pneumoniae serotype 5 (galacturonic acid repeat unit), S. pneumoniae serotype 6A (galactose/glucose/rhamnose repeat unit), S. pneumoniae serotype 6B (galactose/glucose/rhamnose repeat unit), S. pneumoniae serotype 7F (galactose/glucose/rhamnose repeat unit), S. pneumoniae serotype 9V (glucose/galactose/galacturonic acid repeat unit), S. pneumoniae serotype 14 (glucose/galactose repeat unit), S. pneumoniae serotype 18C (galactose/glucose/rhamnose repeat unit), S. pneumoniae serotype 19A (glucose/rhamnose repeat unit), S. pneumoniae serotype 19F (glucose/rhamnose repeat unit), and S. pneumoniae serotype 23F (galactose/rhamnose/glucose repeat unit).

By “source container” (as in saccharide working stock solution source container, saccharide standard receiving container, polysaccharide test sample source container, and polysaccharide test sample receiving container) is intended any receptacle useful for containing a liquid solution during storage and manipulation. Such containers are well known in the art and include, but are not limited to, glass test tubes, plastic test tubes, glass vials, plastic vials, plastic centrifuge tubes, and plastic microcentrifuge tubes.

As used herein, “transferring” (as in transferring a portion of a saccharide working stock solution, transferring a portion of a diluent, transferring a portion of a polysaccharide test sample, transferring a portion of said one or more saccharide standards, transferring a portion of said one or more diluted polysaccharide test samples, and transferring a portion of an acid reagent) includes the conveyance or movement of a liquid from a source container to a receiving container, or the conveyance or movement of a liquid from a source container/receiving container to a well in a multiwell plate. As is well known to one of skill in the art, liquid transference can be accomplished by an automated liquid handling device. Such devices commonly employ one or more multichannel pipette manifolds that allow the transference of multiple liquid samples simultaneously.

By “diluent” is intended an agent used for effecting dilution, for example, of a saccharide working stock solution or a polysaccharide test sample, as well as an agent that serves as a “blank” (i.e., containing no saccharide/polysaccharide) for saccharide concentration determinations that rely on radiant energy absorbance measurements. Diluents that find use in the present invention include, for example, water for injection (WFI), normal saline (i.e., 0.9% NaCl) and succinate-buffered saline. By “mixing” is intended the mingling or blending of the various components in the receiving containers or the wells in a multiwell plate. As is well known to one of skill in the art, the mixing of liquid components can be accomplished by a number of means, including, for example, repetitive pipetting (such as performed by an automated liquid handling device) and shaking (such as performed by a plate shaker).

As used herein, “polysaccharide test sample” includes a sample that contains a polysaccharide(s) of known composition. The composition of the polysaccharide(s) in the test sample must be known, as saccharide concentration is found by determining the amount (e.g., in nanomoles) of saccharide repeat unit present using the equation obtained from the linear regression line produced with absorbance data from the appropriate saccharide standard. Multiplying the molecular weight of a particular serotype polysaccharide repeat unit by the nanomoles of polysaccharide repeat unit obtained from the appropriate linear regression line provides the nanograms of the polysaccharide repeat unit present in the polysaccharide test sample.

Examples of polysaccharides of known composition include bacterial capsular polysaccharides, such as from N. meningitidis serogroup Y, N. meningitidis serogroup W₁₃₅, Group B Streptococcus serotype Ia, Group B Streptococcus serotype III, S. pneumoniae serotype 1, S. pneumoniae serotype 3, S. pneumoniae serotype 4, S. pneumoniae serotype 5, S. pneumoniae serotype 6A, S. pneumoniae serotype 6B, S. pneumoniae serotype 7F, S. pneumoniae serotype 9V, S. pneumoniae serotype 14, S. pneumoniae serotype 18C, S. pneumoniae serotype 19A, S. pneumoniae serotype 19F, and S. pneumoniae serotype 23F. The polysaccharide contained in the test sample can be unmodified, activated (i.e., modified to facilitate its conjugation), conjugated (e.g., to a carrier protein, such as CRM₁₉₇), or any combination thereof. By “diluted polysaccharide test sample” is intended a polysaccharide test sample to which a diluent has been added.

As used herein, “acid reagent” includes a reagent with an acidic pH. In a preferred embodiment, the acid reagent includes sulfuric acid. In some embodiments, in addition to sulfuric acid, the acid reagent includes anthrone. In other embodiments, in addition to sulfuric acid, the acid reagent includes tetraborate.

By “heating said multiwell plate” is intended increasing the temperature of the contents of the wells of the multiwell plate. The combination of an acid pH (i.e., as provided by the acid reagent) and heat breaks down polysaccharides into their constituent monosaccharides. For the methods of the invention, the multiwell plate is heated at a temperature above ambient temperature up to a temperature of 100° C., such as at 75° C., at 80° C., at 85° C., at 90° C., at 95° C., at 96° C., at 97° C., at 98° C., and at 99° C. In a preferred embodiment, the multiwell plate is heated at 95±5° C. for 10±2 minutes. As is well known to one of skill in the art, the amount of time of heating required to effectuate the breakdown of a polysaccharide into its constituent monosaccharides depends on, inter alia, the temperature the polysaccharide is heated at; the lower the temperature, the longer the time of heating that is required. It is within the ability of one of skill in the art to adjust the time of heating based on the temperature used. Heating the multiwell plate can be accomplished by any number of methods well known in the art, such as placing the multiwell plate in a water bath or on a heating plate.

By “cooling said multiwell plate” is intended decreasing the temperature of the contents of the wells of the multiwell plate. Cooling the multiwell plate can be accomplished by any number of methods well known in the art, such as removing the plate from its heat source and allowing it to cool at ambient temperature. Alternatively, the plate can be transferred to an environment (e.g., a refrigerator or a water bath) with a temperature below ambient temperature (such as 4° C.).

As used herein, “shaking said multiwell plate” includes agitating the multiwell plate to mix the contents of its wells. In the methods of the invention, shaking the multiwell plate can be accomplished using any number of devices well known in the art, such as, a plate shaker. By “measuring the absorbance of the contents of all of said series of wells in said multiwell plate” is intended using a spectrophotometer to measure the absorbance of the series of wells containing the blank, the series of wells containing the saccharide standard(s) and the series of wells containing the polysaccharide test sample(s). For polysaccharides that contain neutral hexoses, such as galactose, glucose and rhamnose in their repeat units (e.g., S. pneumoniae serotypes 3, 4, 6A, 6B, 7F, 9V, 14, 18C, 19A, 19F, and 23F; Nm serotypes Y and W₁₃₅; and GBS serotypes Ia and III), the sulfuric acid-anthrone reagent is used and absorbance is determined. In some embodiments, absorbance at 625 nm is determined. For polysaccharides that contain galacturonic acid or glucouronic acid in their repeat units (e.g., S. pneumoniae serotypes 1 and 5), the sulfuric acid-tetraborate reagent along with mHDP is used and absorbance is determined. In some embodiments, absorbance at 520 nm is determined. In the methods of the invention, accuracy is increased by including replicates of the blank, saccharide standard(s) and polysaccharide test sample(s), such as 2, 3, 4, 5, 6, or more replicates. The mean absorbance of a set of replicates for the blank, saccharide standard(s) and polysaccharide test sample(s) are then determined.

In certain embodiments, the method of the present invention includes transferring a portion of a color reagent from a color reagent source container to all of the series of wells in the multiwell plate containing the diluent, the one or more saccharide standards, and the one or more diluted polysaccharide test samples, after cooling the multiwell plate. By “color reagent” is intended a composition that includes a substance that forms a detectable (e.g., spectrophotometrically) color upon reaction with a saccharide. Exemplary color forming substances include anthrone, mHDP and carbazole.

The following examples are offered by way of illustration and not by way of limitation.

### EXPERIMENTAL

### Example 1

### Automated Anthrone Assay for Determination of Saccharide Content

The anthrone assay is used to determine saccharide content for polysaccharides that contain neutral hexoses, such as galactose, glucose and rhamnose in their repeat units (e.g., S. pneumoniae serotypes 3, 4, 6A, 6B, 7F, 9V, 14, 18C, 19A, 19F, and 23F; Nm serotypes Y and W₁₃₅; and GBS serotypes Ia and III). The polysaccharide is broken down into its constituent monosaccharides by the action of sulfuric acid and heat. The anthrone reagent reacts with the hexoses to form a yellow-green colored complex whose absorbance is read spectrophotometrically (e.g., at 625 nm); over the range of the assay, absorbance is proportional to the amount of hexoses present.

### Reagent Preparation

50 mM saccharide stock solutions were prepared using the following formula to calculate the weight of the saccharide required for each serotype stock:

$$mg of saccharide to weigh = \frac{50 mM \times volume ( ml ) \times MW}{1000 ml \text{/} L \times Std . Purity}$$

### Molecular Weights are Shown in Table I.

TABLE 1
Molecular Weights

| | | Galacturonic | | Pyruvic | | N-Acetyl-D- | N-Acetyl-D- |
|---|---|---|---|---|---|---|---|
| Saccharide | Galactose | Acid | Glucose | Acid | Rhamnose | Galactosamine | Mannosamine |
| Molecular | 180.2 | 212.2 | 180.2 | 110.0 | 182.2 | 221.2 | 221.2 |
| Weight | | | | | | | |
| (g/mole) | | | | | | | |

1.0 mM (S. pneumoniae serotypes 3, 4, 6A, 6B, 7F, 9V, 14, 19A, 19F, and 23F, Nm serotypes Y and W₁₃₅, and GBS serotypes Ia and III) and 0.5 mM (S. pneumoniae serotype 18C) saccharide working stock solutions were prepared, using the 50 mM saccharide stock solutions, to reflect the composition of the polysaccharide repeat unit of each serotype (Tables II and III).

TABLE II
S. pneumoniae Saccharide Working Stock Solutions

| Pneumo | | | | | | | | 19A/ | |
|---|---|---|---|---|---|---|---|---|---|
| Serotype(s) | 3 | 4 | 6A/6B | 7F | 9V | 14 | 18C | 19F | 23F |
| Saccharides (50 | Volume (μl) of 50 mM Saccharide Required per Serotype | | | | | | | | |
| mM) | | | | | | | | | |
| Galactose | — | 500 | 500 | 1000 | 500 | 1000 | 500 | — | 500 |
| Galacturonic | — | — | — | — | 500 | — | — | — | — |
| Acid | | | | | | | | | |
| Glucose | 500 | — | 500 | 500 | 1000 | 500 | 1500 | 500 | 500 |
| Pyruvic Acid | — | 500 | — | — | — | — | — | — | — |
| Rhamnose | — | — | 500 | 1000 | — | — | 500 | 500 | 1000 |
| N-Acetyl-D- | — | 1000 | — | — | — | — | — | — | — |
| Galactosamine | | | | | | | | | |
| N-Acetyl-D- | — | 500 | — | — | — | — | — | — | — |
| Mannosamine | | | | | | | | | |
| High Purity | 24.5 | 22.5 | 23.5 | 22.5 | 23.0 | 23.5 | 47.5 | 24.0 | 23.0 |
| Water (ml) | | | | | | | | | |
| Total Volume | 25.0 | 25.0 | 25.0 | 25.0 | 25.0 | 25.0 | 50.0 | 25.0 | 25.0 |
| (ml) | | | | | | | | | |

TABLE III
N. meningitidis/Group B Streptococcus
Saccharide Working Stock Solutions

| Serotype | Nm Y | Nm W₁₃₅ | GBS Ia | GBS III |
|---|---|---|---|---|
| Saccharides (50 | Volume (μl) of 50 mM Saccharide | | | |
| mM) | Required per Serotype | | | |
| Galactose | — | 500 | 1000 | 1000 |
| Galacturonic | — | — | — | — |
| Acid | | | | |
| Glucose | 500 | — | 500 | 500 |
| Pyruvic Acid | — | — | — | — |
| Rhamnose | — | — | — | — |
| N-Acetyl-D- | — | — | — | — |
| Galactosamine | | | | |
| N-Acetyl-D- | — | — | — | — |
| Mannosamine | | | | |
| High Purity | 24.5 | 24.5 | 23.5 | 23.5 |
| Water (ml) | | | | |
| Total Volume | 25.0 | 25.0 | 25.0 | 25.0 |
| (ml) | | | | |

Acid reagent (0.2% anthrone-sulfuric acid) was prepared by mixing 1.0 g of anthrone with 500 ml of sulfuric acid.

### Standard Preparation

For all Nm, GBS and S. pneumoniae serotypes (except 18C), 15 nM, 30 nM, 45 nM, and 60 nM saccharide standards (in 1 ml aliquots) are prepared with a Janus (Perkin-Elmer, Waltham, Mass.) automated liquid handling system in sets of 5 microcentrifuge tubes for each concentration, using the 1.0 mM working stock solution appropriate for the serotype being assayed with WFI as the diluent. For S. pneumoniae serotype 18C, 7.5 nM, 15 nM, 22.5 nM and 30 nM saccharide standards (in 1 ml aliquots) are prepared using the 0.5 mM working stock solution. These volumes are used for preparing a default standard curve.

### Sample Preparation

Dilutions required to bring the sample (e.g., S. pneumoniae serotypes 3, 4, 6A, 6B, 7F, 9V, 14, 18C, 19A, 19F, and 23F; Nm serotypes Y and W₁₃₅; and GBS serotypes Ia and III) into the range of the assay are prepared with a Janus automated liquid handling system in sets of 5 microcentrifuge tubes for each dilution. The following calculation may be used to estimate the amount of sample, for all Nm, GBS and S. pneumoniae serotypes (except 18C):

$$µl sample = \frac{30 nM ( mid \text{-} std . curve ) \times MW}{expected concentration ( mg \text{/} ml ) \times 1000 µg \text{/} mg}$$

$$For 18 C \text{:} µl sample = \frac{15 nM ( mid \text{-} std . curve ) \times MW}{expected concentration ( mg \text{/} ml ) \times 1000 µg \text{/} mg}$$

If the expected concentration of a sample is not well established, a range of dilutions is selected (e.g., 1:50, 1:75, 1:100, 1:150, and 1:200) such that at least one will fall within the range of the assay.

### Procedure

Using the Janus automated liquid handling system with integrated labware movement arm and ancillary instrumentation for reader integration, temperature control and mixing, five replicates at 100 μl of diluent (e.g., WFI) are dispensed into five wells of a multiwell plate; the diluent serving as a blank. Five replicates at 100 μl of each appropriate standard (e.g., S. pneumoniae serotypes 3, 4, 6A, 6B, 7F, 9V, 14, 18C, 19A, 19F, and 23F; Nm serotypes Y and W₁₃₅; or GBS serotypes Ia and III) are dispensed into five wells of the multiwell plate, as are five replicates at 100 μl of each test sample into five wells of the multiwell plate.

Two hundred microliters of acid reagent are added to each well and the contents of the wells mixed thoroughly with repetitive pipetting. The multiwell plate is covered (manually or via the labware movement arm) and heated at 95±5° C. for 10±2 minutes, after which it is cooled at ambient temperature for ≧10 minutes. The multiwell plate is placed on the plate reader by the labware movement arm, gently shaken and radiant energy absorbance of the contents of the wells is determined at 625 nm.

Representative examples of automated runs performed using S. pneumoniae serotype 6A saccharide standards and test samples are shown in Table IV and Table V.

TABLE IV
S. pneumoniae serotype 6A

| | 6A | 6A | 6A | 6A | 6A | 6A | 6A | 6A | 6A |
|---|---|---|---|---|---|---|---|---|---|
| | Stnd. | Stnd. | Stnd. | Stnd. | Sample | Sample | Sample | Sample | Sample |
| Blanks | 15 nM | 30 nM | 45 nM | 60 nM | 1:50 | 1:75 | 1:100 | 1:150 | 1:200 |
| 0.058 | 0.321 | 0.555 | 0.799 | 1.023 | 0.883 | 0.615 | 0.505 | 0.371 | 0.283 |
| 0.067 | 0.351 | 0.577 | 0.828 | 1.020 | 0.909 | 0.646 | 0.527 | 0.390 | 0.288 |
| 0.058 | 0.347 | 0.604 | 0.840 | 1.069 | 0.916 | 0.653 | 0.544 | 0.414 | 0.299 |
| 0.058 | 0.368 | 0.556 | 0.851 | 1.071 | 0.899 | 0.641 | 0.549 | 0.421 | 0.312 |
| 0.087 | 0.350 | 0.548 | 0.807 | 1.044 | 0.869 | 0.616 | 0.524 | 0.402 | 0.301 |

TABLE V
S. pneumoniae serotype 6A

| Dilution | Experiment 1 | Experiment 2 | Experiment 3 | AVG. | Stnd. Dev. | RSD % |
|---|---|---|---|---|---|---|
| 1:15 | 6.2884 | 5.8518 | 6.0099 | 6.050 | 0.221 | 3.65 |
| 1:20 | 6.3441 | 5.6283 | 6.2356 | 6.069 | 0.386 | 6.36 |
| 1:50 | 5.8715 | 5.7002 | 6.1598 | 5.911 | 0.232 | 3.93 |
| AVG. | 6.1680 | 5.7268 | 6.1351 | | | |
| Stnd. Dev. | 0.2583 | 0.1141 | 0.1149 | | | |
| RSD % | 4.1877 | 1.9920 | 1.8725 | | | |
| Dilution | Experiment 4 | Experiment 5 | Experiment 6 | AVG. | Stnd. Dev. | RSD % |
| 1:15 | 5.8135 | 6.0049 | 5.9906 | 5.936 | 0.107 | 1.80 |
| 1:20 | 6.0395 | 6.2118 | 6.1570 | 6.136 | 0.088 | 1.43 |
| 1:50 | 6.0358 | 6.3069 | 6.1604 | 6.168 | 0.136 | 2.20 |
| 1:100 | 5.6668 | 5.8959 | 6.0379 | 5.867 | 0.187 | 3.19 |
| AVG. | 5.9140 | 6.1382 | 6.1184 | | | |
| Stnd. Dev. | 0.2141 | 0.2152 | 0.0698 | | | |
| RSD % | 3.6209 | 3.5055 | 1.1406 | | | |
| Dilution | Experiment 7 | Experiment 8 | Experiment 9 | AVG. | Stnd. Dev. | RSD % |
| 1:50 | 6.0184 | 6.1108 | 6.3469 | 6.159 | 0.169 | 2.75 |
| 1:75 | 6.1008 | 6.0902 | 6.4042 | 6.198 | 0.178 | 2.88 |
| 1:100 | 6.5066 | 6.6051 | 6.7570 | 6.623 | 0.126 | 1.90 |
| AVG. | 6.2086 | 6.2687 | 6.5027 | | | |
| Stnd. Dev. | 0.2613 | 0.2915 | 0.2221 | | | |
| RSD % | 4.2093 | 4.6506 | 3.4149 | | | |
| Grand AVG. | 6.112 | | | | | |
| Experiments 1-9 | | | | | | |
| (n = 30) | | | | | | |
| Grand Stnd. | 0.265 | | | | | |
| Dev. | | | | | | |
| Grand RSD % | 4.34 | | | | | |

Nine experiments were performed using nine different 96-well plates; six replicates of standards and samples were prepared on each plate. For Experiments 1-6, a standard curve was generated using 15 nM, 30 nM and 60 nM standards. For Experiments 7-9, a standard curve was generated using 8 nM, 15 nM, 23 nM, and 30 nM standards. Sample concentrations (mg/ml) are shown. AVG.: average; Stnd. Dev.: standard deviation; RSD: relative standard deviation.

### Example 2

### Automated Uronic Acid Assay for Determination of Saccharide Content

The uronic acid assay is used to determine saccharide content for polysaccharides that contain galacturonic or glucouronic acid monosaccharides in their repeat units (e.g., S. pneumoniae serotypes 1 and 5). The polysaccharide is broken down into its constituent monosaccharides by the action of sulfuric acid, sodium tetraborate and heat. Addition of mHDP causes the formation of a fuchsia-colored complex with absorbance read spectrophotometrically (e.g., at 520 nm); over the range of the assay, absorbance is proportional to the amount of uronic acid present.

### Reagent Preparation

A 50 mM galacturonic acid stock solution was prepared using the following formula to calculate the weight of the galacturonic acid required:

$$mg of galacuronic acid to weigh = \frac{50 mM \times volume ( ml ) \times 212.2 g \text{/} mole}{1000 ml \text{/} L \times Std . Purity}$$

A 1.0 mM galacturonic acid working stock solution was prepared, using the 50 mM galacturonic acid stock solution.

Acid reagent (12.5 mM sodium tetraborate-sulfuric acid) was prepared by mixing 2.38 g of sodium tetraborate decahydrate with 500 ml of sulfuric acid.

### Standard Preparation

Two nanomolar, 4 nM, 8 nM, 12 nM, and 16 nM saccharide standards (in 1 ml aliquots) are prepared with the Janus automated liquid handling system in sets of 5 microcentrifuge tubes for each concentration, using the 1.0 mM galacturonic acid working stock solution with WFI as the diluent. These volumes are used for preparing a default standard curve.

### Sample Preparation

Dilutions required to bring the sample (e.g., S. pneumoniae serotypes 1 and 5) into the range of the assay are prepared with a Janus automated liquid handling system in sets of 5 microcentrifuge tubes for each dilution. The following calculation may be used to estimate the amount of sample, for both S. pneumoniae serotypes:

$$µl sample = \frac{8 nM ( mid \text{-} std . curve ) \times MW}{expected concentration ( mg \text{/} ml ) \times 1000 µg \text{/} mg}$$

If the expected concentration of a sample is not well established, a range of dilutions is selected (e.g., 1:50, 1:75, 1:100, 1:150, and 1:200) such that at least one will fall within the range of the assay.

### Procedure

Using the Janus automated liquid handling system with integrated labware movement arm and ancillary instrumentation for reader integration, temperature control and mixing, five replicates at 40 μl of diluent (e.g., WFI) are dispensed into five wells of a multiwell plate; the diluent serving as a blank. Five replicates at 40 μl of each appropriate standard (e.g., S. pneumoniae serotypes 1 and 5) are dispensed into five wells of the multiwell plate, as are five replicates at 40 μl of each test sample into five wells of the multiwell plate.

Two hundred fifty microliters of acid reagent are added to each well and the contents of the wells mixed thoroughly with repetitive pipetting. The multiwell plate is covered (manually or via the labware movement arm) and heated at 90° C. for 20 minutes, after which it is cooled at ambient temperature for ≧20 minutes. Five microliters of 0.15% mHDP is added to each well and the contents of the wells mixed thoroughly with repetitive pipetting. The contents of the multiwell plate are allowed to react at ambient temperature for 15-20 minutes, after which the multiwell plate is placed on the plate reader by the labware movement arm, gently shaken and radiant energy absorbance of the contents of the wells is determined at 520 nm.

The article “a” and “an” are used herein to refer to one or more than one (i.e., to at least one) of the grammatical object of the article. By way of example, “an element” means one or more element.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. An automated method of determining saccharide concentration, comprising the steps of:
a) preparing one or more saccharide standards, comprising the steps of:
(i) transferring a portion of a saccharide working stock solution from a saccharide working stock solution source container to multiple saccharide standard receiving containers;
(ii) transferring a portion of a diluent from a diluent source container to said multiple saccharide standard receiving containers; and
(iii) mixing the contents of said multiple saccharide standard receiving containers to prepare said one or more saccharide standards;
b) preparing one or more diluted polysaccharide test samples, comprising the steps of:
(i) transferring a portion of a polysaccharide test sample from a polysaccharide test sample source container to multiple polysaccharide test sample receiving containers;
(ii) transferring a portion of the diluent from said diluent source container to said multiple polysaccharide test sample receiving containers; and
(iii) mixing the contents of said multiple polysaccharide test sample receiving containers to prepare said one or more diluted polysaccharide test samples;
c) transferring a portion of the diluent from said diluent source container to a series of wells in a multiwell plate;
d) transferring a portion of said one or more saccharide standards from each of said multiple saccharide standard receiving containers to a series of wells in said multiwell plate;
e) transferring a portion of said one or more diluted polysaccharide test samples from each of said multiple polysaccharide test sample receiving containers to a series of wells in said multiwell plate;
f) transferring a portion of an acid reagent from an acid reagent source container to all of said series of wells in said multiwell plate containing said diluent, said one or more saccharide standards, and said one or more diluted polysaccharide test samples;
g) mixing the contents of all of said series of wells in said multiwell plate containing said diluent, said one or more saccharide standards, and said one or more diluted polysaccharide test samples;
h) covering said multiwell plate with a cover;
i) heating said multiwell plate;
j) cooling said multiwell plate;
k) shaking said multiwell plate; and
l) measuring the radiant energy absorbance of the contents of all of said series of wells in said multiwell plate containing said diluent, said one or more saccharide standards, and said one or more diluted polysaccharide test samples, wherein steps a-1 are performed without human intervention.

2. The method of claim 1, further comprising the step of transferring a portion of a color reagent from a color reagent source container to all of said series of wells in said multiwell plate containing said diluent, said one or more saccharide standards, and said one or more diluted polysaccharide test samples, after cooling said multiwell plate.

3. The method of claim 2, wherein said color reagent comprises meta-hydroxydiphenyl.

4. The method of claim 1, wherein said saccharide working stock solution comprises one or more saccharides selected from the group consisting of rhamnose, galactose, glucose, galacturonic acid, pyruvic acid, N-acetyl-D-galactosamine, and N-acetyl-D-mannosamine.

5. The method of claim 1, wherein said polysaccharide test sample comprises one or more of a bacterial capsular polysaccharide, an activated bacterial capsular polysaccharide or a bacterial capsular polysaccharide-protein conjugate.

6. The method of claim 5, wherein said bacterial capsular polysaccharide comprises a bacterial capsular polysaccharide selected from a group of bacteria consisting of N. meningitidis serogroup Y, N. meningitidis serogroup W₁₃₅, Group B Streptococcus serotype Ia, Group B Streptococcus serotype III, S. pneumoniae serotype 1, S. pneumoniae serotype 3, S. pneumoniae serotype 4, S. pneumoniae serotype 5, S. pneumoniae serotype 6A, S. pneumoniae serotype 6B, S. pneumoniae serotype 7F, S. pneumoniae serotype 9V, S. pneumoniae serotype 14, S. pneumoniae serotype 18C, S. pneumoniae serotype 19A, S. pneumoniae serotype 19F, S. pneumoniae serotype 23F, and combinations thereof.

7. The method of claim 1, wherein said acid reagent comprises sulfuric acid and anthrone.

8. The method of claim 2, wherein said acid reagent comprises sulfuric acid and tetraborate.

9. The method of claim 1, wherein said saccharide working stock solution source containers, said saccharide standard receiving containers, said polysaccharide test sample source containers, and said polysaccharide test sample receiving containers are selected from the group consisting of glass test tubes, plastic test tubes, glass vials, plastic vials, plastic centrifuge tubes, plastic microcentrifuge tubes, and combinations thereof.

10. The method of claim 1, wherein said diluent is selected from the group consisting of water for injection, normal saline, succinate-buffered saline, and combinations thereof.

11. The method of claim 1, wherein said diluent is water for injection.

12. The method of claim 1, wherein said transferring is performed by an automated liquid handling device.

13. The method of claim 12, wherein said automated liquid handling device comprises a multichannel pipette manifold.

14. The method of claim 1, wherein said mixing is performed by repetitive pipetting.

15. The method of claim 1, wherein heating said multiwell plate is performed by a plate heater.

16. An automated method of determining saccharide concentration, comprising the steps of:
a) preparing one or more saccharide standards, comprising the steps of:
(i) transferring a portion of a saccharide working stock solution comprising one or more saccharides selected from the group consisting of rhamnose, galactose, glucose, galacturonic acid, pyruvic acid, N-acetyl-D-galactosamine, and N-acetyl-D-mannosamine from a saccharide working stock solution source container to multiple saccharide standard receiving containers;
(ii) transferring a portion of a diluent from a diluent source container to said multiple saccharide standard receiving containers; and
(iii) mixing the contents of said multiple saccharide standard receiving containers to prepare said one or more saccharide standards;
b) preparing one or more diluted polysaccharide test samples, comprising the steps of:
(i) transferring a portion of a polysaccharide test sample comprising a bacterial capsular polysaccharide selected from a group of bacteria consisting of S. pneumoniae serotypes 3, 4, 6A, 6B, 7F, 9V, 14, 18C, 19A, 19F, and 23F from a polysaccharide test sample source container to multiple polysaccharide test sample receiving containers;
(ii) transferring a portion of the diluent from said diluent source container to said multiple polysaccharide test sample receiving containers; and
(iii) mixing the contents of said multiple polysaccharide test sample receiving containers to prepare said one or more diluted polysaccharide test samples;
c) transferring a portion of the diluent from said diluent source container to a series of wells in a multiwell plate;
d) transferring a portion of said one or more saccharide standards from each of said multiple saccharide standard receiving containers to a series of wells in said multiwell plate;
e) transferring a portion of said one or more diluted polysaccharide test samples from each of said multiple polysaccharide test sample receiving containers to a series of wells in said multiwell plate;
f) transferring a portion of a sulfuric acid-anthrone reagent from a sulfuric acid-anthrone reagent source container to all of said series of wells in said multiwell plate containing said diluent, said one or more saccharide standards, and said one or more diluted polysaccharide test samples;
g) mixing the contents of all of said series of wells in said multiwell plate containing said diluent, said one or more saccharide standards, and said one or more diluted polysaccharide test samples;
h) covering said multiwell plate with a cover;
i) heating said multiwell plate;
j) cooling said multiwell plate;
k) shaking said multiwell plate; and
l) measuring the radiant energy absorbance of the contents of all of said series of wells in said multiwell plate containing said diluent, said one or more saccharide standards, and said one or more diluted polysaccharide test samples, wherein steps a-1 are performed without human intervention.

17. The method of claim 16, wherein said bacterial capsular polysaccharide selected from a group of bacteria consisting of S. pneumoniae serotypes 3, 4, 6A, 6B, 7F, 9V, 14, 18C, 19A, 19F, and 23F is an activated bacterial capsular polysaccharide.

18. The method of claim 16, wherein said bacterial capsular polysaccharide selected from a group of bacteria consisting of S. pneumoniae serotypes 3, 4, 6A, 6B, 7F, 9V, 14, 18C, 19A, 19F, and 23F is conjugated to a carrier protein.

19. The method of claim 18, wherein said carrier protein is CRM₁₉₇.

20. An automated method of determining saccharide concentration, comprising the steps of:
a) preparing one or more saccharide standards, comprising the steps of:
(i) transferring a portion of a saccharide working stock solution comprising galacturonic acid from a saccharide working stock solution source container to multiple saccharide standard receiving containers;
(ii) transferring a portion of a diluent from a diluent source container to said multiple saccharide standard receiving containers; and
(iii) mixing the contents of said multiple saccharide standard receiving containers to prepare said one or more saccharide standards;
b) preparing one or more diluted polysaccharide test samples, comprising the steps of:
(i) transferring a portion of a polysaccharide test sample comprising a bacterial capsular polysaccharide selected from a group of bacteria consisting of S. pneumoniae serotypes 1 and 5 from a polysaccharide test sample source container to multiple polysaccharide test sample receiving containers;
(ii) transferring a portion of the diluent from said diluent source container to said multiple polysaccharide test sample receiving containers; and
(iii) mixing the contents of said multiple polysaccharide test sample receiving containers to prepare said one or more diluted polysaccharide test samples;
c) transferring a portion of the diluent from said diluent source container to a series of wells in a multiwell plate;
d) transferring a portion of said one or more saccharide standards from each of said multiple saccharide standard receiving containers to a series of wells in said multiwell plate;
e) transferring a portion of said one or more diluted polysaccharide test samples from each of said multiple polysaccharide test sample receiving containers to a series of wells in said multiwell plate;
f) transferring a portion of a sulfuric acid-tetraborate reagent from a sulfuric acid-tetraborate reagent source container to all of said series of wells in said multiwell plate containing said diluent, said one or more saccharide standards, and said one or more diluted polysaccharide test samples;
g) mixing the contents of all of said series of wells in said multiwell plate containing said diluent, said one or more saccharide standards, and said one or more diluted polysaccharide test samples;
h) covering said multiwell plate with a cover;
i) heating said multiwell plate;
j) cooling said multiwell plate;
k) transferring a portion of a color reagent comprising meta-hydroxydiphenyl from a color reagent source container to all of said series of wells in said multiwell plate containing said diluent, said one or more saccharide standards, and said one or more diluted polysaccharide test samples;
l) shaking said multiwell plate; and
m) measuring the radiant energy absorbance of the contents of all of said series of wells in said multiwell plate containing said diluent, said one or more saccharide standards, and said one or more diluted polysaccharide test samples,
wherein steps a-m are performed without human intervention.

21. The method of claim 20, wherein said bacterial capsular polysaccharide selected from a group of bacteria consisting of S. pneumoniae serotypes 1 and 5 is an activated bacterial capsular polysaccharide.

22. The method of claim 20, wherein said bacterial capsular polysaccharide selected from a group of bacteria consisting of S. pneumoniae serotypes 1 and 5 is conjugated to a carrier protein.

23. The method of claim 22, wherein said carrier protein is CRM₁₉₇.
